(19) United States
(12) Patent Application Publication
(10) Pub. No.: US 2004/0101504 A1
(43) Pub. Date: May 27, 2004

(54) Mild antibacterial liquid dish cleaning composition having improved stability

(75) Inventors: Kevin Kinscherf, Freehold, NJ (US); Evangelia Arvanitidou, Princeton, NJ (US)

Correspondence Address: Patent Department, Colgate-Palmolive Company, 909 River Road, P.O. Box 1343, Piscataway, NJ, 08855-1343, US

(73) Assignee: Colgate-Palmolive Company

(21) Appl. No.: 10/702,783

(22) Filed: Nov. 6, 2003

Related U.S. Application Data
(63) Continuation-in-part of application No. 10/350,717, filed on Jan. 27, 2003.
Application No. 10/350,717 is a continuation-in-part of application No. 10/085,556, filed on Feb. 27, 2002, now Pat. No. 6,583,178.
Application No. 10/085,556 is a continuation-in-part of application No. 09/853,791, filed on May 11, 2001, now Pat. No. 6,441,037.

(51) Int. Cl. A61K 7/075; A61K 7/08

(52) U.S. Cl. 424/70.24

(57) ABSTRACT

An antibacterial liquid dish cleaning composition with desirable cleansing properties comprising a C₈₋₁₈ ethoxylated alkyl ether sulfate, two anionic surfactant, a betaine surfactant, a hydroxy containing organic acid, an alkyl polyglucoside surfactant, a preservative, at least one solubilizer and water.

### RELATED APPLICATION

This application is a continuation in part application of U.S. Ser. No. 10/350,717 filed Jan. 27, 2003 which in turn is a continuation in part application of U.S. Ser. No. 10/085,556 filed Feb. 27, 2002 which in turn is a continuation in part application of U.S. Ser. No. 9/853,791 filed May 11, 2001.

### FIELD OF INVENTION

This invention relates to a mild antibacterial liquid dish cleaning composition of high active surfactant level containing surfactants and peroxide encapsulated beads, having improved stability which is designed to disinfect the surface being treated while maintaining good foaming grease cutting, rinsing and mildness properties.

### BACKGROUND OF THE INVENTION

The present invention relates to novel light duty liquid detergent compositions containing surfactants and peroxide encapsulated beads with an improved stability profile and high foaming and good grease cutting properties as well as disinfecting properties.

The prior art is replete with light duty liquid detergent compositions containing nonionic surfactants in combination with anionic and/or betaine surfactants wherein the nonionic detergent is not the major active surfactant. In U.S. Pat. No. 3,658,985 an anionic based shampoo contains a minor amount of a fatty acid alkanolamide. U.S. Pat. No. 3,769,398 discloses a betaine-based shampoo containing minor amounts of nonionic surfactants. This patent states that the low foaming properties of nonionic detergents renders its use in shampoo compositions non-preferred. U.S. Pat. No. 4,329,335 also discloses a shampoo containing a betaine surfactant as the major ingredient and minor amounts of a nonionic surfactant and of a fatty acid mono- or di-ethanolamide. U.S. Pat. No. 4,259,204 discloses a shampoo comprising 0.8 to 20% by weight of an anionic phosphoric acid ester and one additional surfactant which may be either anionic, amphoteric, or nonionic. U.S. Pat. No. 4,329,334 discloses an anionic-amphoteric based shampoo containing a major amount of anionic surfactant and lesser amounts of a betaine and nonionic surfactants.

U.S. Pat. No. 3,935,129 discloses a liquid cleaning composition containing an alkali metal silicate, urea, glycerin, triethanolamine, an anionic detergent and a nonionic detergent. The silicate content determines the amount of anionic and/or nonionic detergent in the liquid cleaning composition. However, the foaming properties of these detergent compositions are not discussed therein.

U.S. Pat. No. 4,129,515 discloses a heavy duty liquid detergent for laundering fabrics comprising a mixture of substantially equal amounts of anionic and nonionic surfactants, alkanolamines and magnesium salts, and, optionally, zwitterionic surfactants as suds modifiers.

U.S. Pat. No. 4,224,195 discloses an aqueous detergent composition for laundering socks or stockings comprising a specific group of nonionic detergents, namely, an ethylene oxide of a secondary alcohol, a specific group of anionic detergents, namely, a sulfuric ester salt of an ethylene oxide adduct of a secondary alcohol, and an amphoteric surfactant which may be a betaine, wherein either the anionic or nonionic surfactant may be the major ingredient.

The prior art also discloses detergent compositions containing all nonionic surfactants as shown in U.S. Pat. Nos. 4,154,706 and 4,329,336 wherein the shampoo compositions contain a plurality of particular nonionic surfactants in order to affect desirable foaming and detersive properties despite the fact that nonionic surfactants are usually deficient in such properties.

U.S. Pat. No. 4,013,787 discloses a piperazine based polymer in conditioning and shampoo compositions which may contain all nonionic surfactant or all anionic surfactant.

U.S. Pat. No. 4,450,091 discloses high viscosity shampoo compositions containing a blend of an amphoteric betaine surfactant, a polyoxybutylenepolyoxyethylene nonionic detergent, an anionic surfactant, a fatty acid alkanolamide and a polyoxyalkylene glycol fatty ester. But, none of the exemplified compositions contain an active ingredient mixture wherein the nonionic detergent is present in major proportion which is probably due to the low foaming properties of the polyoxybutylene polyoxyethylene nonionic detergent.

U.S. Pat. No. 4,595,526 describes a composition comprising a nonionic surfactant, a betaine surfactant, an anionic surfactant and a C₁₂-C₁₄ fatty acid monoethanolamide foam stabilizer.

U.S. Pat. No. 6,147,039 teaches an antibacterial hand cleaning composition having a low surfactant content.

### SUMMARY OF THE INVENTION

It has now been found that a mild, antibacterial liquid dish cleaning composition containing peroxide encapsulated beads and having improved stability can be formulated with three different anionic surfactants, an amine oxide surfactant, an ethoxylated nonionic surfactant, an alkyl glucoside surfactant, a solubilizer, a preservative, and water which has desirable cleaning and foaming properties.

An object of this invention is to provide an antibacterial liquid dish cleaning composition which comprises a sulfate surfactant, two sulfonate anionic surfactants, an amine oxide surfactant, an ethoxylated nonionic surfactant, an alkyl polyglucoside surfactant, a solubilizer, peroxide encapsulated beads, a preservative and water, wherein the composition does not contain any silicas, abrasives, acyl isoethionate, 2-hydroxy-4,2′,4′-trichloridiphenyl ether, phosphoric acid, phosphonic acid, boric acid, alkali metal carbonates, alkaline earth metal carbonates, alkyl glycine surfactant, cyclic imidinium surfactant, or more than 3 wt. % of a fatty acid or salt thereof.

Another object of this invention is to provide a mild antibacterial liquid dish cleaning composition having improved stability and with desirable high foaming and cleaning properties which kills bacteria.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### DETAILED DESCRIPTION OF THE INVENTION

This invention relates to an antibacterial liquid dish cleaning composition which comprises approximately by weight:

(a) 1% to 9% of a sodium salt of a C₈-C₁₆ linear alkyl benzene sulfonate surfactant;

(b) 3% to 16% of a magnesium salt of a C₈-C₁₆ linear alkyl benzene sulfonate surfactant;

(c) 4% to 16% of an ammonium or sodium salt of an ethoxylated C₈-C₁₈ alkyl ether sulfate surfactant;

(d) 0.1% to 6% of an amine oxide surfactant;

(e) 0.1% to 5%, more preferably 0.5% to 4% of an encapsulated peroxide bead;

(f) 2% to 12% of an alkyl polyglucoside surfactant;

(g) 0.1% to 10%, more preferably 0.5% to 10% of at least one solubilizer;

(h) 0.005% to 3.0% of a preservative;

(i) 8 to 20% of an ethoxylated nonionic surfactant; and

(j) the balance being color, fragrance, UV absorber and water, wherein the composition has a pH of 3 to 4.5 and has a viscosity of 200 to 800 cps, more preferably 200 to 600 cps at 25° C. using a #21 spindle at 20 rpm as measured on a Brookfield RVTDV-II viscometer, wherein the composition does not contain any grease release agents such as choline chloride or buffering system which is a nitrogenous buffer which is ammonium or alkaline earth carbonate, zwitterionic surfactants, guanidine derivates, alkoxylalkyl amines and alkyleneamines C₃-C₇ alkyl and alkenyl monobasic and dibasic acids such as C₄-C₇ aliphatic carboxylic diacids which do not contain a hydroxy group, boric acid, phosphoric acid, hydrogen peroxide, peracetic acid and amino alkylene phosphonic acid.

The anionic sulfonate surfactants which may be used in the detergent of this invention are selected from the consisting of water soluble and include the sodium, potassium, ammonium, magnesium and ethanolammonium salts of linear C₈-C₁₆ alkyl benzene sulfonates; C₁₀-C₂₀ paraffin sulfonates, alpha olefin sulfonates containing about 10-24 carbon atoms and C₈-C₁₈ alkyl sulfates and mixtures thereof.

The paraffin sulfonates may be monosulfonates or di-sulfonates and usually are mixtures thereof, obtained by sulfonating paraffins of 10 to 20 carbon atoms. Preferred paraffin sulfonates are those of C₁₂₋₁₈ carbon atoms chains, and more preferably they are of C₁₄₋₁₇ chains. Paraffin sulfonates that have the sulfonate group(s) distributed along the paraffin chain are described in U.S. Pat. Nos. 2,503,280; 2,507,088; 3,260,744; and 3,372,188; and also in German Patent 735,096. Such compounds may be made to specifications and desirably the content of paraffin sulfonates outside the C₁₄₋₁₇ range will be minor and will be minimized, as will be any contents of di- or poly-sulfonates.

Examples of suitable other sulfonated anionic detergents are the well known higher alkyl mononuclear aromatic sulfonates, such as the higher alkylbenzene sulfonates containing 9 to 18 or preferably 9 to 16 carbon atoms in the higher alkyl group in a straight or branched chain, or C₈₋₁₅ alkyl toluene sulfonates. A preferred alkylbenzene sulfonate is a linear alkylbenzene sulfonate having a higher content of 3-phenyl (or higher) isomers and a correspondingly lower content (well below 50%) of 2-phenyl (or lower) isomers, such as those sulfonates wherein the benzene ring is attached mostly at the 3 or higher (for example 4, 5, 6 or 7) position of the alkyl group and the content of the isomers in which the benzene ring is attached in the 2 or 1 position is correspondingly low. Preferred materials are set forth in U.S. Pat. No. 3,320,174, especially those in which the alkyls are of 10 to 13 carbon atoms.

The C₈₋₁₈ ethoxylated alkyl ether sulfate surfactants have the structure

R—(OCHCH₂)ₙOSO₃⁻M⁺

wherein n is about 1 to about 22 more preferably 1 to 3 and R is an alkyl group having about 8 to about 18 carbon atoms, more preferably 12 to 15 and natural cuts, for example, C₁₂₋₁₄ or C₁₂₋₁₆ and M is an ammonium cation or a metal cation, most preferably sodium.

The ethoxylated alkyl ether sulfate may be made by sulfating the condensation product of ethylene oxide and C₈₋₁₀ alkanol, and neutralizing the resultant product. The ethoxylated alkyl ether sulfates differ from one another in the number of carbon atoms in the alcohols and in the number of moles of ethylene oxide reacted with one mole of such alcohol. Preferred ethoxylated alkyl ether polyethenoxy sulfates contain 12 to 15 carbon atoms in the alcohols and in the alkyl groups thereof, e.g., sodium myristyl (3 EO) sulfate.

Ethoxylated C₈₋₁₈ alkylphenyl ether sulfates containing from 2 to 6 moles of ethylene oxide in the molecule are also suitable for use in the invention compositions. These detergents can be prepared by reacting an alkyl phenol with 2 to 6 moles of ethylene oxide and sulfating and neutralizing the resultant ethoxylated alkylphenol. The concentration of the ethoxylated alkyl ether sulfate surfactant is about 1 to about 8 wt. %.

Amine oxide semi-polar nonionic surfactants comprise compounds and mixtures of compounds having the formula

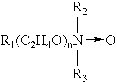

wherein R₁ is an alkyl, 2-hydroxyalkyl, 3-hydroxyalkyl, or 3-alkoxy-2-hydroxypropyl radical in which the alkyl and alkoxy, respectively, contain from 8 to 18 carbon atoms, R₂ and R₃ are each methyl, ethyl, propyl, isopropyl, 2-hydroxyethyl, 2-hydroxypropyl, or 3-hydroxypropyl, and n is from 0 to 10. Particularly preferred are amine oxides of the formula:

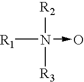

wherein R₁ is a C₁₂₋₁₆ alkyl and R₂ and R₃ are methyl or ethyl. The above ethylene oxide condensates, amides, and amine oxides are more fully described in U.S. Pat. No. 4,316,824 which is hereby incorporated herein by reference.

The water soluble aliphatic ethoxylated nonionic surfactants utilized in this invention are commercially well known and include the primary aliphatic alcohol ethoxylates and secondary aliphatic alcohol ethoxylates. The length of the polyethenoxy chain can be adjusted to achieve the desired balance between the hydrophobic and hydrophilic elements.

The nonionic surfactant class includes the condensation products of a higher alcohol (e.g., an alkanol containing about 8 to 16 carbon atoms in a straight or branched chain configuration) condensed with about 1 to 20 moles of ethylene oxide, for example, lauryl or myristyl alcohol condensed with about 16 moles of ethylene oxide (EO), tridecanol condensed with about 6 to 15 moles of EO, myristyl alcohol condensed with about 10 moles of EO per mole of myristyl alcohol, the condensation product of EO with a cut of coconut fatty alcohol containing a mixture of fatty alcohols with alkyl chains varying from 10 to about 14 carbon atoms in length and wherein the condensate contains either about 6 moles of EO per mole of total alcohol or about 9 moles of EO per mole of alcohol and tallow alcohol ethoxylates containing 6 EO to 11 EO per mole of alcohol.

A preferred group of the foregoing nonionic surfactants are the Neodol ethoxylates (Shell Co.), which are higher aliphatic, primary alcohol containing about 9-15 carbon atoms, such as C₉-C₁₁ alkanol condensed with 1 to 10 moles of ethylene oxide (Neodol 91-8 or Neodol 91-5), C₁₂₋₁₃ alkanol condensed with 6.5 moles ethylene oxide (Neodol 23-6.5), C₁₂₋₁₅ alkanol condensed with 12 moles ethylene oxide (Neodol 25-12), C₁₄₋₁₅ alkanol condensed with 13 moles ethylene oxide (Neodol 45-13), and the like. Such ethoxamers have an HLB (hydrophobic lipophilic balance) value of about 8 to 15 and give good O/W emulsification, whereas ethoxamers with HLB values below 7 contain less than 4 ethyleneoxide groups and tend to be poor emulsifiers and poor detergents.

Additional satisfactory water soluble alcohol ethylene oxide condensates are the condensation products of a secondary aliphatic alcohol containing 8 to 18 carbon atoms in a straight or branched chain configuration condensed with 5 to 30 moles of ethylene oxide. Examples of commercially available nonionic detergents of the foregoing type are C₁₁-C₁₅ secondary alkanol condensed with either 9 EO (Tergitol 15-S-9) or 12 EO (Tergitol 15-S-12) marketed by Union Carbide.

The water soluble ethoxylated/propoxylated nonionic surfactants which can be utilized in this invention are an aliphatic ethoxylated/propoxylated nonionic surfactants which are depicted by the formula:

R—O—(CH₂CH₂O)ₓ—(CH₂CH₂CH₂O)_{y}—H

or

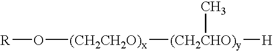

wherein R is a branched chain alkyl group having about 10 to about 16 carbon atoms, preferably an isotridecyl group and x and y are independently numbered from 1 to 20. A preferred ethoxylated/propoxylated nonionic surfactant is Plurafac® 300 manufactured by BASF.

The peroxide encapsulated beads are an organic peroxide such as benzoyl peroxide, urea peroxide or carbamide encapsulated in gelatin or arabic gum. The internal phase of the bead comprises 0.5 wt. % to 8 wt. % of the organic peroxide and 92 wt. % to 99.5 wt. % of mineral oil. The beads comprises 56 wt. % to 58 wt. % of wall of the bead which is gelatin or arabic gum and 37 wt. % to 41 wt. % external aqueous phase containing water, xanthan and a preservative, wherein the bead exists as a slurry in the aqueous external phase.

The alkyl polysaccharides surfactants, which are used in conjunction with the aforementioned surfactants have a hydrophobic group containing from about 8 to about 20 carbon atoms, preferably from about 10 to about 16 carbon atoms, most preferably from about 12 to about 14 carbon atoms, and polysaccharide hydrophilic group containing from about 1.5 to about 10, preferably from about 1.5 to about 4, most preferably from about 1.6 to about 2.7 saccharide units (e.g., galactoside, glucoside, fructoside, glucosyl, fructosyl; and/or galactosyl units). Mixtures of saccharide moieties may be used in the alkyl polysaccharide surfactants. The number x indicates the number of saccharide units in a particular alkyl polysaccharide surfactant. For a particular alkyl polysaccharide molecule x can only assume integral values. In any physical sample of alkyl polysaccharide surfactants there will be in general molecules having different x values. The physical sample can be characterized by the average value of x and this average value can assume non-integral values. In this specification the values of x are to be understood to be average values. The hydrophobic group (R) can be attached at the 2-, 3-, or 4-positions rather than at the 1-position, (thus giving e.g. a glucosyl or galactosyl as opposed to a glucoside or galactoside). However, attachment through the 1-position, i.e., glucosides, galactoside, fructosides, etc., is preferred. In the preferred product the additional saccharide units are predominately attached to the previous saccharide unit's 2-position. Attachment through the 3-, 4-, and 6-positions can also occur. Optionally and less desirably there can be a polyalkoxide chain joining the hydrophobic moiety (R) and the polysaccharide chain. The preferred alkoxide moiety is ethoxide.

Typical hydrophobic groups include alkyl groups, either saturated or unsaturated, branched or unbranched containing from about 8 to about 20, preferably from about 10 to about 18 carbon atoms. Preferably, the alkyl group is a straight chain saturated alkyl group. The alkyl group can contain up to 3 hydroxy groups and/or the polyalkoxide chain can contain up to about 30, preferably less than about 10, alkoxide moieties.

Suitable alkyl polysaccharides are decyl, dodecyl, tetradecyl, pentadecyl, hexadecyl, and octadecyl, di-, tri-, tetra-, penta-, and hexaglucosides, galactosides, lactosides, fructosides, fructosyls, lactosyls, glucosyls and/or galactosyls and mixtures thereof.

The alkyl monosaccharides are relatively less soluble in water than the higher alkyl polysaccharides. When used in admixture with alkyl polysaccharides, the alkyl monosaccharides are solubilized to some extent. The use of alkyl monosaccharides in admixture with alkyl polysaccharides is a preferred mode of carrying out the invention. Suitable mixtures include coconut alkyl, di-, tri-, tetra-, and pentaglucosides and tallow alkyl tetra-, penta-, and hexaglucosides.

The preferred alkyl polysaccharides are alkyl polyglucosides having the formula

R₂O(CₙH₂ₙO)r(Z)ₓ

wherein Z is derived from glucose, R is a hydrophobic group selected from the group consisting of alkyl, alkylphenyl, hydroxyalkylphenyl, and mixtures thereof in which said alkyl groups contain from about 10 to about 18, preferably from about 12 to about 14 carbon atoms; n is 2 or 3 preferably 2, r is from 0 to 10, preferable 0; and x is from 1.5 to 8, preferably from 1.5 to 4, most preferably from 1.6 to 2.7. To prepare these compounds a long chain alcohol (R₂OH) can be reacted with glucose, in the presence of an acid catalyst to form the desired glucoside. Alternatively the alkyl polyglucosides can be prepared by a two step procedure in which a short chain alcohol (R₁OH) can be reacted with glucose, in the presence of an acid catalyst to form the desired glucoside. Alternatively the alkyl polyglucosides can be prepared by a two step procedure in which a short chain alcohol (C₁₋₆) is reacted with glucose or a polyglucoside (x=2 to 4) to yield a short chain alkyl glucoside (x=1 to 4) which can in turn be reacted with a longer chain alcohol (R₂OH) to displace the short chain alcohol and obtain the desired alkyl polyglucoside. If this two step procedure is used, the short chain alkylglucosde content of the final alkyl polyglucoside material should be less than 50%, preferably less than 10%, more preferably less than about 5%, most preferably 0% of the alkyl polyglucoside.

The amount of unreacted alcohol (the free fatty alcohol content) in the desired alkyl polysaccharide surfactant is preferably less than about 2%, more preferably less than about 0.5% by weight of the total of the alkyl polysaccharide. For some uses it is desirable to have the alkyl monosaccharide content less than about 10%.

The used herein, “alkyl polysaccharide surfactant” is intended to represent both the preferred glucose and galactose derived surfactants and the less preferred alkyl polysaccharide surfactants. Throughout this specification, “alkyl polyglucoside” is used to include alkyl polyglycosides because the stereochemistry of the saccharide moiety is changed during the preparation reaction.

An especially preferred APG glycoside surfactant is APG 625 glycoside manufactured by the Henkel Corporation of Ambler, Pa. APG25 is a nonionic alkyl polyglycoside characterized by the formula:

CₙH₂ₙ₊₁O(C₆H₁₀O₅)ₓH

wherein n=10 (2%); n=122 (65%); n=14 (21-28%); n=16 (4-8%) and n=18 (0.5%) and x (degree of polymerization)=1.6. APG 625 has: a pH of 6 to 10 (10% of APG 625 in distilled water); a specific gravity at 25° C. of 1.1 g/ml; a density at 25° C. of 9.1 lbs/gallon; a calculated HLB of 12.1 and a Brookfield viscosity at 35° C., 21 spindle, 5-10 RPM of 3,000 to 7,000 cps.

The hydroxy containing organic acid is ortho hydroxy benzoic acid or preferably a hydroxy aliphatic acid selected from the group consisting of lactic acid, citric acid, salicylic acid and glycolic and mixtures thereof, wherein citric acid is preferred.

The instant light duty liquid nonmicroemulsion compositions can contain about 0 wt. % to about 10 wt. %, more preferably about 1 wt. % to about 8 wt. %, of at least one solubilizing agent selected from the group consisting of a C₂₋₅ mono, dihydroxy or polyhydroxy alkanols such as ethanol, isopropanol, glycerol ethylene glycol, diethylene glycol, propylene glycol, and hexylene glycol and mixtures thereof and alkali metal cumene or xylene sulfonates such as sodium cumene sulfonate and sodium xylene sulfonate. The solubilizing agents are included in order to control low temperature cloud clear properties.

The instant formulas explicitly exclude alkali metal silicates and alkali metal builders such as alkali metal polyphosphates, alkali metal carbonates, alkali metal phosphonates and alkali metal citrates because these materials, if used in the instant composition, would cause the composition to have a high pH as well as leaving residue on the surface being cleaned.

The final essential ingredient in the inventive compositions having improved interfacial tension properties is water. The proportion of water in the compositions generally is in the range of 10% to 95%.

The liquid cleaning composition of this invention may, if desired, also contain other components either to provide additional effect or to make the product more attractive to the consumer. The following are mentioned by way of example: Colors or dyes in amounts up to 0.5% by weight; bactericides in amounts up to 1% by weight; UV absorbents, or antioxidizing agents, and pH adjusting agents, such as sulfuric acid or sodium hydroxide, as needed. Furthermore, if opaque compositions are desired, up to 4% by weight of an opacifier may be added.

Preservatives which can be used in the instant compositions at a concentration of 0.005 wt. % to 3 wt. %, more preferably 0.01 wt. % to 2.5 wt. % are: benzalkonium chloride; benzethonium chloride, 5-bromo-5-nitro-1,3dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N′-(hydroxy methyl) urea; 1-3-dimethyol-5, 5-dimethyl hydantoin; formaldehyde; iodopropynl butyl carbamata, butyl paraben; ethyl paraben; methyl paraben; propyl paraben, mixture of methyl isothiazolinone/methyl-chloroisothiazoline in a 1:3 wt. ratio; mixture of phenoxythanol/butyl paraben/methyl paraben/propylparaben; 2-phenoxyethanol; tris-hydroxyethyl-hexahydrotriazine; methylisothiazolinone; 5-chloro-2-methyl-4-isothiazolin-3-one; 1,2-dibromo-2, 4-dicyanobutane; 1-(3-chloroalkyl)-3,5,7-triaza-azoniaadamantane chloride; and sodium benzoate. PH adjusting agents such as sulfuric acid or sodium hydroxide can be used as needed.

In final form, the instant compositions exhibit stability at reduced and increased temperatures. More specifically, such compositions remain clear and stable in the range of 0° C. to 50° C., especially 5° C. to 43° C. Such compositions exhibit a pH of 3 to 5.

The following examples illustrate the liquid body cleaning compositions of the described invention. Unless otherwise specified, all percentages are by weight. The exemplified compositions are illustrative only and do not limit the scope of the invention. Unless otherwise specified, the proportions in the examples and elsewhere in the specification are by weight.

The instant compositions have a minimum foam volume of 350 mls after 40 rotations at room temperature as measured by the foam volume test using 0.0333 wt. % of the Ultra composition in 150 ppm of water. The Shake foam test is an inverted cylinder test in which 100 gr. of a 0.0333 wt. % LDL formula in 150 ppm of H₂O is placed in a stoppered graduate cylinder (500 ml) and inverted 40 cycles at a rate of 30 cycles/minute. After 40 inversions, the foam volume which has been generated is measured in mls inside the graduated cylinder. This value includes the 100 ml of LDL solution inside the cylinder. After the initial volume is measured, 175 microliters of whole milk is added to the solution. The cylinder is then inverted for another 40 cycles and a foam volume with soil is measured; the foam volume after the soil addition is at least 130 mls. The values provided above include the 100 ml's of LDL solution inside the cylinder.

The Modified Germicidal Spray Test was used to determine the surface disinfection profile. The Modified Germicidal Spray test protocol was designed by MicroBiotest Inc., Sterling Va., to determine percentage of germs killed on hard surfaces such as dishware. The method determines the efficacy of products intended to be used for one-step cleaning and germ killing on surface of dishware and is based on the Germicidal Spray Products test, Official Methods of Analysis, Sixteenth edition, 1995, AOAC.

### EXAMPLE 1

The following composition is listed as a wt. %. The physical properties and appearance (i.e. viscosity, cloud point, color) are unchanged upon aging in extreme temperature conditions.

| | A | B |
|---|---|---|
| Magnesium linear alkyl benzene sulfonate | 6.31 | 6.31 |
| Sodium linear alkyl benzene sulfonate | 2.1 | 2.1 |
| Ammonium alkyl ether sulfate 1.3 EO | 8.05 | 8.05 |
| Alkyl polyglucoside | 6.65 | 6.65 |
| Neodol 1-3 | 15 | 15 |
| Lauryl myristyl amine oxide | 3.5 | 3.5 |
| Fragrance | 0.4 | 0.4 |
| Ethanol | 0.88 | 0.88 |
| Benzyoyl peroxide beads | 5 | 2 |
| Glucoserve LAD preservative | 0.08 | 0.08 |
| Benzotriazolyl Dodecyle P-Cresol, | 0.03 | 0.03 |
| Dodecene Homopolymer | | |
| (Tinogard TL)- UV absorber | | |
| Deionized water | Balance | Balance |
| pH | 6.8 | 6.8 |
| Separationof peroxide beads upon | 3 | 0 |
| aging (0 = none, 5 = severe) | | |

### EXAMPLE 2

The following composition is listed as a wt. %. The dishwashing performance as shown below foam measurements, has been unchanged upon aging at extreme temperature conditions.

| | A |
|---|---|
| Magnesium linear alkyl benzene sulfonate | 9 |
| Sodium linear alkyl benzene sulfonate | 3 |
| Ammonium alkyl ether sulfate 1.3EO | 11.5 |
| Akyl polyglucoside | 9.5 |
| Citric acid | 1 |
| Cocoamidopropyl betaine | 5 |
| Fragrance | 1 |
| Ethanol | 1.3 |
| Sodium xylene sulfonate | 3.5 |
| Orange color solution | 0.14 |
| Glucoserve LAD preservative | 0.11 |
| Perfume | 0.45 |
| Benzotriazolyl Dodecyle P- Cresol, | 0.03 |
| Dodecene Homopolymer | |
| (Tinogard TL)- UV absorber | |
| Deionized water | Balance |
| pH | 3.5 |
| Initial Performance: Shake foam, I (ml) | 377 |
| Initial Performance: Shake foam, F (ml) | 143 |
| Performance after 2 weeks @ 135F: | 365 |
| Shake foam, I (ml) | |
| Performance after 2 weeks @ 135F: | 140 |
| Shake foam, F (ml) | |
| Modified GST (1% dilution, 1 minute contact time/ | >=2 log |
| staph aureus and e-coli or | |
| salmonella)/ Log reduction | |

What is claimed:

1. A mild antibacterial liquid dish cleaning composition which comprises approximately by weight:
(a) 1% to 9% of a sodium salt of a C₈-C₁₆ linear alkyl benzene sulfonate surfactant;
(b) 4% to 16% of a magnesium salt of a C₈-C₁₆ linear alkyl benzene sulfonate surfactant;
(c) 4% to 16% of an ammonium or sodium salt of an ethoxylated C₈-C₁₈ alkyl ether sulfate surfactant;
(d) 0.1% to 5% of a zwitterionic surfactant;
(e) 0.5% to 5% of a hydroxy containing organic acid;
(f) 5% to 15% of an alkyl polyglucoside surfactant;
(g) 0.1% to 10% of at least one solubilizer;
(h) 0.005% to 3.0% of a preservative; and
(i) the balance being color, perfume, UV absorber and water, wherein the composition has a pH of 3 to 4 and has a viscosity of 100 to 1,000 cps at 25° C. using a #21 spindle at 20 rpm as measured on a Brookfield RVTDV-11 viscometer, wherein the composition does not contain any choline chloride ammonium or alkaline earth carbonate, amine oxide surfactants, guanidine derivates, alkoxylalkyl amines and alkyleneamines C₃-C₇ alkyl and alkenyl monobasic and dibasic acids such as C₄-C₇ aliphatic carboxylic diacids which do not contain a hydroxy group, boric acid, phosphoric acid, ethoxylated nonionic surfactants, and amino alkylene phosphonic acid and the composition is pourable and not a gel has a complex viscosity at 1 rads⁻¹ of less than 0.4 Pascal seconds.
